(19) United States
(12) Patent Application Publication
(10) Pub. No.: US 2021/0386643 A1
(43) Pub. Date: Dec. 16, 2021

(54) EFFECTIVE TEETH WHITENING COMPOSITION UTILIZING BROMELAIN ENZYMES

(71) Applicant: Amarillo Junior College District, Amarillo, TX (US)

(72) Inventors: Asanga Devinda Ranasinghe, Amarillo, TX (US); Cecilia Rizcallah, Amarillo, TX (US)

(73) Assignee: Amarillo Junior College District, Amarillo, TX (US)

(21) Appl. No.: 17/344,572

(22) Filed: Jun. 10, 2021

Related U.S. Application Data
(60) Provisional application No. 63/037,042, filed on Jun. 10, 2020.

(51) Int. Cl. A61K 8/66; A61Q 11/00; A61K 8/34; A61K 8/19; A61K 8/73; A61K 8/46; A61K 8/21

(52) U.S. Cl. CPC A61K 8/66; A61Q 11/00; C12Y 304/22032; A61K 8/345; A61K 2800/92; A61K 8/73; A61K 8/463; A61K 8/21; A61K 2800/87; A61K 8/19

(57) ABSTRACT

The present invention embodies an effective teeth whitening composition that utilizes bromelain enzymes as the primary active ingredient and the main source of whitening. Further, the invention is a method for preparing the effective teeth whitening composition. The composition of an effective teeth whitener includes a sugar alcohol, calcium carbonate, a binder, an anionic detergent, a fluoride agent, sodium benzoate, and bromelain.

### CROSS-REFERENCE TO RELATED APPLICATIONS

This application claims priority to U.S. Provisional Patent Application Ser. No. 63/037,042, filed Jun. 10, 2020, entitled “Effective Teeth Whitener Made Up Entirely With Bromelain Enzymes.” This patent application is commonly assigned to the Assignee of the present invention and is incorporated herein by reference in its entirety for all purposes.

### TECHNICAL FIELD

The present invention relates to an effective teeth whitening composition that can whiten teeth using bromelain as both an active ingredient and the main ingredient that whitens teeth. More particularly, the effective teeth whitening composition of the invention includes a sugar alcohol, calcium carbonate, a binder, sodium laurel sulfate, a fluoride agent, sodium benzoate, and bromelain. Additionally, the present invention relates to a method for preparing the effective teeth whitening composition.

### STATEMENT OF FEDERALLY FUNDED RESEARCH

None.

### BACKGROUND OF THE DISCLOSURE

In conventional practice, peroxide and peroxyacid compounds, such as hydrogen peroxide and carbamide peroxide, are utilized as active ingredients within teeth whitening compositions. In fact, the majority of existing teeth whiteners on the market use hydrogen peroxide and sodium bicarbonate as additives. But these chemicals can often cause severe complications when used within whitening compositions. Specifically, many whitening treatments using such active ingredients can lead to sensitivity of the teeth and decaying of the gum line.

Bromelain—a protein-digesting enzyme mixture derived from the pineapple plant—does not harm the teeth and surrounding gums in the way that the leading active ingredients in teeth whitening compositions have been known to do. Bromelain is also relatively low cost of an ingredient for commercial compositions. But, since bromelain is an enzyme and is therefore susceptible to oxidization, it has not been utilized in teeth whitening compositions. Further, the pH range of commercially available teeth whitening compositions are often incapable of activing enzymes and would result in a highly unstable composition if an enzyme, such as bromelain, were introduced.

Since molecules have a high oxidative potential, the use of enzymes has not been explored in teeth whitening compositions. Despite the recent understanding that bromelain does not harm the teeth in the way of most active ingredients in teeth whitening compositions, the concern of instability that would lead to even greater oxidation of tooth enamel has been prohibitive to using bromelain in the field of teeth whitening compositions.

Thus, traditional whitening compositions for teeth and general dental care are still used even though they can be harsh on the teeth; the teeth can be damaged using teeth whitening compositions because the low pH can result in an oxidative process on the enamel. From this, traditional teeth whitening compositions have garnered a reputation for resulting in sensitivity and pain. Moreover, long-lasting and severe gum damage may arise from repeat use of traditional teeth whitening compositions. Accordingly, there is a need for a teeth whitening composition that is capable of whitening teeth without oxidizing the enamel, decaying the gum line. There is also a need for stability in teeth whitening compositions to prevent damage from additional oxidation.

It is therefore the objective of the present invention to introduce a stable and effective teeth whitening composition using an active ingredient of bromelain as means to provide a low cost, enzyme-based alternative. Such an alternative being useful to whiten teeth compared to the common treatments found in the teeth whitening market, as described through the method for preparing the effective teeth whitening composition.

### SUMMARY OF THE DISCLOSURE

The present invention is directed to an effective teeth whitening composition and a method for preparing the effective teeth whitening composition. The present invention provides a solution to at least of the aforementioned problems in the prior art. In one aspect, the present invention utilizes bromelain to provide a less evasive method for whitening teeth. Through the use of bromelain combined with the other components of the mixture, the present invention satisfies the need for an effective, low cost, and stable teeth whitening composition without complications of increasing teeth sensitivity or decaying the gum line. When utilizing the mixture of the composition and following the method of preparation of the invention, the pH range of the bromelain allows the enzyme to maintain an active effect and efficiency. Thus, the present invention provides a stable composition for teeth whitening, which overcomes the stability concerns associated with using bromelain in isolation from the other components of the composition or in a manner outside of the method of preparation.

In general, in one embodiment, the invention features an effective teeth whitening composition including a sugar alcohol, where between 20 wt % and 40 wt % of the composition is the sugar alcohol. The composition also includes calcium carbonate, where between 15 wt % and 20 wt % of the composition is the calcium carbonate. The composition also includes a binder, where between 0.25 wt % and 2.5 wt % of the composition is the binder. The composition also includes an anionic detergent, where between 0.5 wt % and 2.5 wt % of the composition is the anionic detergent. The composition also includes a fluoride agent, where between 0.1 wt % and 0.3 wt % of the composition is the fluoride agent. The composition also includes sodium benzoate, where between 0.15 wt % and 0.25 wt % of the composition is the sodium benzoate. The composition also includes bromelain, where between 0.5 wt % and 15 wt % of the composition is the bromelain, and the composition has a pH in a range between 7 and 10.

Implementations of the invention can include one or more of the following features:

The sugar alcohol can be incapable of metabolizing into glucose.

The sugar alcohol can be selected from a group consisting of sorbitol, xylitol, or combinations thereof.

The sugar alcohol can be sorbitol.

The calcium carbonate can be finely granulated.

The binder can be selected from a group consisting of xanthan gum, guar gum, carrageegan, acacia gum, hydroxyethyl cellulose, or combinations thereof.

The binder can be xanthan gum.

The anionic detergent can be selected from a group consisting of sodium lauryl sulfate, ethylenediaminetetraacetic acid (EDTA), surfactants, or combinations thereof.

The anionic detergent can be sodium lauryl sulfate.

The fluoride agent can be selected from a group consisting of stannous fluoride, sodium fluoride, hydrofluoric acid, acidulated fluorophosphate, or combinations thereof.

The fluoride agent can be stannous fluoride.

The composition can also include glycerin.

In general, in another embodiment, the invention features a method for preparing an effective teeth whitening composition. The method includes selecting ingredients including a sugar alcohol, calcium carbonate, a binder, an anionic detergent, a fluoride agent, sodium benzoate, and bromelain. The method further includes combining the ingredients in a vessel. The method further includes mixing the ingredients in the vessel to form a mixture. The mixture having between 20 wt % and 40 wt % of the mixture being the sugar alcohol. The mixture also having between 15 wt % and 50 wt % of the mixture being calcium carbonate. The mixture also having between 0.25 wt % and 2.5 wt % of the mixture being the binder. The mixture also having between 0.5 wt % and 2.5 wt % of the mixture being the anionic detergent. The mixture also having between 0.1 wt % and 0.3 wt % of the mixture being the fluoride agent. The mixture also having between 0.15 wt % and 0.25 wt % of the mixture being sodium benzoate. The mixture also having between 0.5 wt % and 15 wt % of the mixture being bromelain. The mixture also having a pH in a range between 3 and 10. The method further includes cooling the mixture in the vessel, where the pH of the mixture is in a range between 7 and 10.

Implementations of the invention can include one or more of the following features:

The mixture can be a gel, a paste, or a liquid formulation.

Responsive to cooling the mixture in the vessel, the method can include loading the mixture into a tray, where the tray is fitted for a mouth.

Prior to mixing the ingredients in the vessel to form a mixture, the method can include adding glycerin to the vessel.

Responsive to cooling the mixture in the vessel, the method can include introducing a neutral liquid to the mixture. The introduction of the neutral liquid can form a paste.

Responsive to cooling the mixture in the vessel, the method can include storing the mixture in a chilled location.

The sugar alcohol can be selected from a group consisting of sorbitol, xylitol, or combinations thereof.

The binder can be selected from a group consisting of xanthan gum, guar gum, carrageegan, acacia gum, hydroxyethyl cellulose, or combinations thereof.

The anionic detergent can be selected from a group consisting of sodium lauryl sulfate, ethylenediaminetetraacetic acid (EDTA), surfactants, or combinations thereof.

The fluoride agent can be selected from a group consisting of stannous fluoride, sodium fluoride, hydrofluoric acid, acidulated fluorophosphate, or combinations thereof.

### BRIEF DESCRIPTION OF THE DRAWINGS

The following drawings form part of the present specification and are included to further demonstrate certain non-limiting aspects of the present invention. Thus, some embodiments of the present invention are illustrated as an example and are not limited by the figures of the accompanying drawings. Other advantages of the present invention will be apparent from the following detailed description of the invention in conjunction with embodiments as illustrated in the accompanying drawings, in which:

FIG. 1A depicts a perspective frontal view of a stained tooth.

FIG. 1B depicts a perspective frontal view of the stained tooth after being subjected to exposure of the invention in its preferred form.

FIG. 2A depicts a perspective frontal view of a stained tooth.

FIG. 2B depicts a perspective frontal view of the stained tooth after being subjected to exposure of a hydrogen peroxide treatment.

FIG. 3 depicts a flow diagram illustrating a method for preparing the effective teeth whitening composition, in accordance with the embodiments of this disclosure.

### DETAILED DESCRIPTION OF THE DISCLOSURE

While the making and using of various embodiments of the present invention are discussed in detail below, it should be appreciated that the present invention provides many applicable inventive concepts that can be embodied in a wide variety of specific contexts, goods, or services. The specific embodiments discussed herein are merely illustrative of specific ways to make and use the disclosure and do not delimit the scope of the disclosure.

All publications and patent applications mentioned in the specification are indicative of the level of skill of those skilled in the art to which this disclosure pertains. All publications and patent applications are herein incorporated by reference to the same extent as if each individual publication or patent application was specifically and individually indicated to be incorporated by reference.

The present invention will now be described more fully hereinafter with reference to the accompanying drawings, which form a part hereof, and which show, by way of illustration, specific example embodiments. Subject matter may, however, be embodied in a variety of different forms and, therefore, covered or claimed subject matter is intended to be construed as not being limited to any example embodiments set forth herein; example embodiments are provided merely to be illustrative. Likewise, a reasonably broad scope for claimed or covered subject matter is intended. Among other things, for example, subject matter may be embodied as methods, devices, components, or systems. The following detailed description is, therefore, not intended to be taken in a limiting sense.

Throughout the specification and claims, terms may have nuanced meanings suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter include combinations of example embodiments in whole or in part.

In general, terminology may be understood at least in part from usage in context. For example, terms, such as “and”, “or”, or “and/or,” as used herein may include a variety of meanings that may depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” as used herein, depending at least in part upon context, may be used to describe any feature, structure, or characteristic in a singular sense or may be used to describe combinations of features, structures or characteristics in a plural sense. Similarly, terms, such as “a,” “an,” or “the,” again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

The present invention relates to an effective teeth whitening composition, and further, the method for preparing the effective teeth whitening composition. The effective teeth whitening composition utilizes bromelain—an enzyme—as the active ingredient and primary source of whitening.

In the preferred embodiment, the effective teeth whitening composition includes a sugar alcohol. In such an embodiment, the sugar alcohol can be selected from any sugar that is either incapable of metabolizing into glucose or has not yet metabolized into glucose in the composition. This is because metabolizing into glucose would be harmful to the enamel of the teeth. Thus, in certain embodiments, the sugar alcohol can be selected from a group that consists of sorbitol, xylitol, or some combination thereof. In the embodiment of the trials for the effective teeth whitening composition, the sugar alcohol that was utilized was sorbitol because it is non-toxic to pets and humans. Using a sugar alcohol similar to sorbitol allows the mouth to retain moisture during the whitening process. Further, sorbitol includes less bacteria than other similar sugar alcohols.

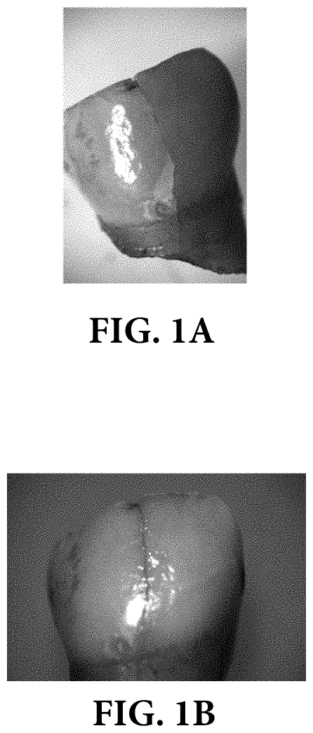

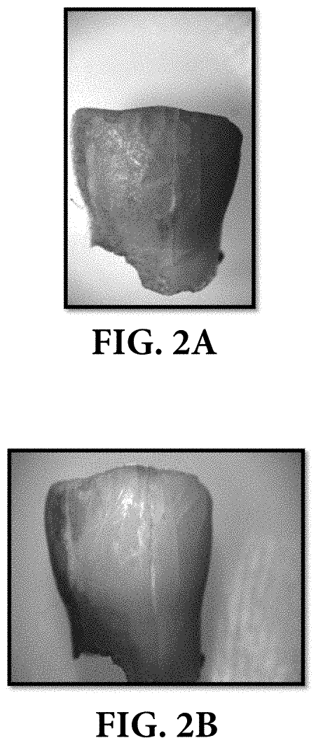

Additionally, in the preferred embodiment, the effective teeth whitening composition includes calcium carbonate. In some embodiments, magnesium carbonate could be utilized along with or in place of the calcium carbonate. Further, in the exemplary embodiments that were utilized to produce the results displayed in FIGS. 1B and 2B, the calcium carbonate was finely granulated. Calcium carbonate is cost effective, does not pose a toxicity problem when used orally, and keeps the pH of the composition buffered. However, calcium carbonate can also act as an abrasive. Thus, in some embodiments, the calcium carbonate can be finely granulated as a means to reduce abrasive tendencies and to facilitate the incorporation of the calcium carbonate into the composition. In some embodiments, the finely granulated calcium carbonate is between 30 and 150 mesh.

In the preferred embodiment, the effective teeth whitening composition includes a binder. The binder acts as a means to improve the texture and consistency of the composition. Beyond this, in the present invention, the binder acts in part to stabilize the effective teeth whitening composition by helping the composition to stabilize at particular temperatures and pH values. Since the present invention is an effective teeth whitening composition and is basic in pH, the binder helps to stabilize the pH in a range between 7 and 10. In some embodiments, the binder can be selected from a group consisting of xanthan gum, guar gum, carrageegan, acacia gum, hydroxyethyl cellulose, or combinations thereof. In the exemplary embodiment used to produce the results in FIGS. 1B and 2B, the binder was xanthan gum.

Additionally, in the preferred embodiment, the effective teeth whitening composition includes an anionic detergent, which acts to make the whitening composition turn to a foam or have a foam-like consistency during the application process. In certain embodiments, the anionic detergent can be selected from a group consisting of sodium lauryl sulfate, ethylenediaminetetraacetic acid (EDTA), surfactants, or combinations thereof. In the exemplary embodiment used to produce the results in FIGS. 1B and 2B, the anionic detergent was sodium lauryl sulfate.

In the preferred embodiment, the effective teeth whitening composition includes a fluoride agent. Fluoride agents are widely utilized within the teeth whitening, cleaning, and protecting markets due to the fluoride ions ability to act as a preventative for cavities. Accordingly, in some embodiments, the bound form of the fluoride ion within the fluoride agent allows for activity within the surface of the tooth. In certain embodiments, the fluoride agent could be replaced with a nitrite. In other embodiments, fluoride agent can be selected from a group consisting of stannous fluoride, sodium fluoride, hydrofluoric acid, acidulated fluorophosphate, or combinations thereof. In the exemplary embodiment used to produce the results in FIGS. 1B and 2B, the fluoride agent was stannous fluoride, which also was capable of acting as a protectant against the sensitivity that is commonly associated with teeth whitening processes.

Moreover, in the preferred embodiment, the effective teeth whitening composition includes sodium benzoate. In such an embodiment, the sodium benzoate acts as a preservative and pH stabilizer.

In the preferred embodiment, the effective teeth whitening composition includes bromelain. Because enzymes are oxidative and unstable, prior art has avoided the use of enzymes in teeth whitening compositions. However, in the present invention, bromelain as an enzyme effective acts as the main component for whitening. The reason the present invention is capable of utilizing this ingredient—despite the inability shown through the prior art—is because of the relatively gentle enzyme activity within bromelain and the stability induced within the composition by the other components and the method of preparation.

More specifically, enzyme are inherently structurally instable. Enzyme active sites are capable of producing specific conditions, but as a result are highly susceptible to influences from the surrounding environment. An increasing surrounding temperature will increase the reaction rate of the enzyme, but reducing the temperature beyond a certain point will often reduce the efficacy of the enzyme. Bromelain enzymes, when chilled to a refrigerated temperature, increases in stability and neither metabolizes nor oxidizes. Furthermore, in the present invention the weight percentages of the ingredients are optimized to ensure that the pH is controlled to additionally heighten the stability of the composition.

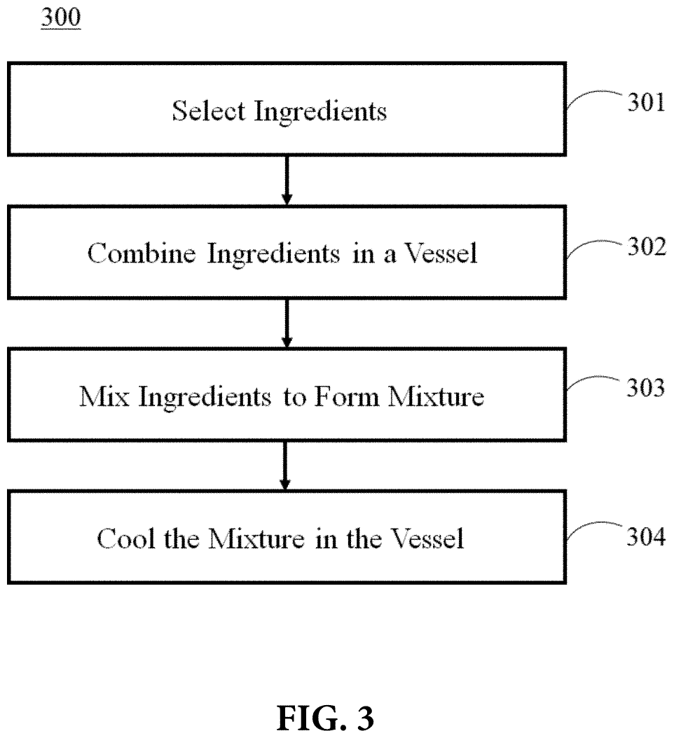

FIG. 3 depicts a flow diagram of an illustrative process for preparing an effective teeth whitening composition 300, according to some embodiments. As shown in FIG. 3, method 300 beings at step 301. At step 301, the preparation is begun by selecting ingredients. The ingredients, in this embodiment, include a sugar alcohol, calcium carbonate, a binder, an anionic detergent, a fluoride agent, sodium benzoate, and bromelain.

Following the selection of step 301, in some embodiments, as shown in FIG. 3, step 302 continues the preparation of the effective teeth whitening composition by combining the ingredients in a vessel. In certain embodiments, the vessel may be a mixing bowl, a tray, or a cooled mixing vessel.

At step 303, the preparation method 300 continues with mixing the ingredients in the vessel to form a mixture. In some embodiments, the mixture has a composition of: between 20 wt % and 40 wt % of the mixture is a sugar alcohol, between 15 wt % and 50 wt % of the mixture is calcium carbonate, between 0.25 wt % and 2.5 wt % of the mixture is a binder, between 0.5 wt % and 2.5 wt % of the mixture is an anionic detergent, between 0.1 wt % and 0.3 wt % of the mixture is a fluoride agent, between 0.15 wt % and 0.25 wt % of the mixture is sodium benzoate, and between 0.5 wt % and 15 wt % of the mixture is bromelain. Further, in certain embodiments, during the mixing of step 303, the mixture can have a pH in a range between 3 and 10. In some embodiments prior to the start of step 303, glycerin is added to the combined ingredients.

At step 304, the preparation method 300 follows with cooling the mixture in the vessel, wherein the pH of the mixture is in a range between 7 and 10. In some embodiments, responsive to cooling the mixture in the vessel in step 304, the preparation continues with a step involving loading the mixture into a tray. In such an embodiment, the tray is fitted for a mouth. In some embodiments, responsive to cooling the mixture in the vessel in step 304, the preparation continues with a step involving introducing a neutral liquid to the mixture. In such an embodiment, the introduction of the neutral liquid forms a paste. In such an embodiment, the neutral liquid can be any liquid that does not change the pH of the composition and does not render the bromelain instable. In some embodiments, responsive to cooling the mixture in the vessel in step 304, the preparation continues with a step involving storing the mixture in a chilled location.

Once the method 300 is completed, the effective teeth whitening composition is prepared and ready for a user to use orally. FIG. 1A depicts a perspective frontal view of a stained tooth, which is capable of being treated with a teeth whitening composition. FIG. 1A, specifically, shows a baby tooth that has been immersed in coffee for an hour prior to being subjected to exposure of whitening treatments. FIG. 1B depicts a perspective frontal view of the stained tooth after being subjected to exposure of the invention in its preferred form for 10 minutes. As shown by FIGS. 1A and 1B, the baby tooth has reduced staining after being treated by the present invention.

FIG. 2A depicts a perspective frontal view of a stained tooth, which is capable of being treated with a teeth whitening composition. FIG. 2A, specifically, shows a baby tooth that has been immersed in coffee for an hour prior to being subjected to exposure of whitening treatments. FIG. 2B depicts a perspective frontal view of the stained tooth after being subjected to a hydrogen peroxide treatment for 10 minutes. FIG. 2B also depicts that the baby tooth has reduced staining after being treated by a hydrogen peroxide treatment. The tooth in FIG. 2B displays no greater signs of whitening than the tooth in FIG. 1B, but the tooth in FIG. 2B would be susceptible to greater sensitivity and enamel decay.

Thus, because of the equivalent whitening power and the reduced chance of harm, there is a benefit to whitening treatments using the effective teeth whitening composition. In some embodiments, the effective teeth whitening composition can be applied as a gel for 20 minutes intervals. In such an embodiment, this application could be done three to five times in a succession with two-minute rest intervals. Following such an embodiment, users could repeat this process three times for a total of nine rounds of gel application to obtain the desired result of whitening.

Consistent with the above disclosure, the examples of systems and method enumerated in the following clauses are specifically contemplated and are intended as a non-limiting set of examples.

Clause 1. An effective teeth whitening composition comprising: a sugar alcohol, wherein between 20 wt % and 40 wt % of the composition is the sugar alcohol; calcium carbonate, wherein between 15 wt % and 50 wt % of the composition is the calcium carbonate; a binder, wherein between 0.25 wt % and 2.5 wt % of the composition is the binder; an anionic detergent, wherein between 0.5 wt % and 2.5 wt % of the composition is the anionic detergent; a fluoride agent, wherein between 0.1 wt % and 0.3 wt % of the composition is the fluoride agent; sodium benzoate, wherein between 0.15 wt % and 0.25 wt % of the composition is the sodium benzoate; and bromelain, wherein between 0.5 wt % and 15 wt % of the composition is the bromelain, and the composition has a pH in a range between 7 and 10.

Clause 2. The composition of any foregoing clause, wherein the sugar alcohol in the composition has not metabolized into glucose.

Clause 3. The composition of any foregoing clause, wherein the sugar alcohol is selected from a group consisting of sorbitol, xylitol, or combinations thereof.

Clause 4. The composition of Clause 3, wherein the sugar alcohol is sorbitol.

Clause 5. The composition of any foregoing clause, wherein the calcium carbonate is finely granulated.

Clause 6. The composition of any foregoing clause, wherein the binder is selected from a group consisting of xanthan gum, guar gum, carrageegan, acacia gum, hydroxyethyl cellulose, or combinations thereof.

Clause 7. The composition of Clause 6, wherein the binder is xanthan gum.

Clause 8. The composition of any foregoing clause, wherein the anionic detergent is selected from a group consisting of sodium lauryl sulfate, ethylenediaminetetraacetic acid (EDTA), surfactants, or combinations thereof.

Clause 9. The composition of Clause 8, wherein the anionic detergent is sodium lauryl sulfate.

Clause 10. The composition of any foregoing clause, wherein the fluoride agent is selected from a group consisting of stannous fluoride, sodium fluoride, hydrofluoric acid, acidulated fluorophosphate, or combinations thereof.

Clause 11. The composition of Clause 10, wherein the fluoride agent is stannous fluoride.

Clause 12. The composition of any foregoing clause further comprising glycerin.

Clause 13. A method for preparing an effective teeth whitening composition, the method comprising: selecting ingredients comprising a sugar alcohol, calcium carbonate, a binder, an anionic detergent, a fluoride agent, sodium benzoate, and bromelain; combining the ingredients in a vessel; mixing the ingredients in the vessel to form a mixture, wherein: between 20 wt % and 40 wt % of the mixture is a sugar alcohol, between 15 wt % and 50 wt % of the mixture is calcium carbonate, between 0.25 wt % and 2.5 wt % of the mixture is a binder, between 0.5 wt % and 2.5 wt % of the mixture is an anionic detergent, between 0.1 wt % and 0.3 wt % of the mixture is a fluoride agent, between 0.15 wt % and 0.25 wt % of the mixture is sodium benzoate, between 0.5 wt % and 15 wt % of the mixture is bromelain, and the mixture has a pH in a range between 3 and 10; cooling the mixture in the vessel, wherein the pH of the mixture is in a range between 7 and 10.

Clause 14. The method of any foregoing clause, wherein the mixture is a gel, a paste, or a liquid formulation.

Clause 15. The method of any foregoing clause further comprising, responsive to cooling the mixture in the vessel, loading the mixture into a tray, wherein the tray is fitted for a mouth.

Clause 16. The method of any foregoing clause further comprising, prior to mixing the ingredients in the vessel to form a mixture, adding glycerin to the vessel.

Clause 17. The method of any foregoing clause further comprising, responsive to cooling the mixture in the vessel, introducing a neutral liquid to the mixture, wherein the introduction of the neutral liquid forms a paste.

Clause 18. The method of any foregoing clause further comprising, responsive to cooling the mixture in the vessel, storing the mixture in a chilled location.

Clause 19. The composition of any foregoing clause, wherein the sugar alcohol is selected from a group consisting of sorbitol, xylitol, or combinations thereof.

Clause 20. The composition of any foregoing clause, wherein the binder is selected from a group consisting of xanthan gum, guar gum, carrageegan, acacia gum, hydroxyethyl cellulose, or combinations thereof.

Clause 21. The composition of any foregoing clause, wherein the anionic detergent is selected from a group consisting of sodium lauryl sulfate, ethylenediaminetetraacetic acid (EDTA), surfactants, or combinations thereof.

Clause 22. The composition of any foregoing clause, wherein the fluoride agent is selected from a group consisting of stannous fluoride, sodium fluoride, hydrofluoric acid, acidulated fluorophosphate, or combinations thereof.

Additional variations of these embodiments will be obvious to those of ordinary skill in the art. Therefore, the spirit and scope of the appended claims should not be limited to the foregoing description. Only those claims specifically reciting “means for” or “step for” should be construed in the manner required under the sixth paragraph of 35 U.S.C. § 112.

Those skilled in the art will recognize that the methods and systems of the present invention may be implemented in many manners and as such are not to be limited by the foregoing exemplary embodiments and examples. Furthermore, the embodiments of methods presented and described as flowcharts in this disclosure are provided by way of example in order to provide a more complete understanding of the technology. The disclosed methods are not limited to the operations and logical flow presented herein. Alternative embodiments are contemplated in which the order of the various operations is altered and in which sub-operations described as being part of a larger operation are performed independently.

What is claimed is:

1. An effective teeth whitening composition comprising:
(a) a sugar alcohol, wherein between 20 wt % and 40 wt % of the composition is the sugar alcohol;
(b) calcium carbonate, wherein between 15 wt % and 50 wt % of the composition is the calcium carbonate;
(c) a binder, wherein between 0.25 wt % and 2.5 wt % of the composition is the binder;
(d) an anionic detergent, wherein between 0.5 wt % and 2.5 wt % of the composition is the anionic detergent;
(e) a fluoride agent, wherein between 0.1 wt % and 0.3 wt % of the composition is the fluoride agent;
sodium benzoate, wherein between 0.15 wt % and 0.25 wt % of the composition is the sodium benzoate; and
(g) bromelain, wherein
(i) between 0.5 wt % and 15 wt % of the composition is the bromelain, and
(ii) the composition has a pH in a range between 7 and 10.

2. The composition of claim 1, wherein the sugar alcohol in the composition has not metabolized into glucose.

3. The composition of claim 1, wherein the sugar alcohol is selected from a group consisting of sorbitol, xylitol, or combinations thereof.

4. The composition of claim 3, wherein the sugar alcohol is sorbitol.

5. The composition of claim 1, wherein the calcium carbonate is finely granulated.

6. The composition of claim 1, wherein the binder is selected from a group consisting of xanthan gum, guar gum, carrageegan, acacia gum, hydroxyethyl cellulose, or combinations thereof.

7. The composition of claim 6, wherein the binder is xanthan gum.

8. The composition of claim 1, wherein the anionic detergent is selected from a group consisting of sodium lauryl sulfate, ethylenediaminetetraacetic acid (EDTA), surfactants, or combinations thereof.

9. The composition of claim 8, wherein the anionic detergent is sodium lauryl sulfate.

10. The composition of claim 1, wherein the fluoride agent is selected from a group consisting of stannous fluoride, sodium fluoride, hydrofluoric acid, acidulated fluorophosphate, or combinations thereof.

11. The composition of claim 10, wherein the fluoride agent is stannous fluoride.

12. The composition of claim 1 further comprising glycerin.

13. A method for preparing an effective teeth whitening composition, the method comprising:
(a) selecting ingredients comprising a sugar alcohol, calcium carbonate, a binder, an anionic detergent, a fluoride agent, sodium benzoate, and bromelain;
(b) combining the ingredients in a vessel;
(c) mixing the ingredients in the vessel to form a mixture, wherein:
(i) between 20 wt % and 40 wt % of the mixture is a sugar alcohol,
(ii) between 15 wt % and 50 wt % of the mixture is calcium carbonate,
(iii) between 0.25 wt % and 2.5 wt % of the mixture is a binder,
(iv) between 0.5 wt % and 2.5 wt % of the mixture is an anionic detergent,
(v) between 0.1 wt % and 0.3 wt % of the mixture is a fluoride agent,
(vi) between 0.15 wt % and 0.25 wt % of the mixture is sodium benzoate,
(vii) between 0.5 wt % and 15 wt % of the mixture is bromelain, and
(viii) the mixture has a pH in a range between 3 and 10;
(d) cooling the mixture in the vessel, wherein the pH of the mixture is in a range between 7 and 10.

14. The method of claim 13, wherein the mixture is a gel, a paste, or a liquid formulation.

15. The method of claim 13 further comprising, responsive to cooling the mixture in the vessel, loading the mixture into a tray, wherein the tray is fitted for a mouth.

16. The method of claim 13 further comprising, prior to mixing the ingredients in the vessel to form a mixture, adding glycerin to the vessel.

17. The method of claim 13 further comprising, responsive to cooling the mixture in the vessel, introducing a neutral liquid to the mixture, wherein the introduction of the neutral liquid forms a paste.

18. The method of claim 13 further comprising, responsive to cooling the mixture in the vessel, storing the mixture in a chilled location.

19. The composition of claim 13, wherein the sugar alcohol is selected from a group consisting of sorbitol, xylitol, or combinations thereof.

20. The composition of claim 13, wherein the binder is selected from a group consisting of xanthan gum, guar gum, carrageegan, acacia gum, hydroxyethyl cellulose, or combinations thereof.

21. The composition of claim 13, wherein the anionic detergent is selected from a group consisting of sodium lauryl sulfate, ethylenediaminetetraacetic acid (EDTA), surfactants, or combinations thereof.

22. The composition of claim 13, wherein the fluoride agent is selected from a group consisting of stannous fluoride, sodium fluoride, hydrofluoric acid, acidulated fluorophosphate, or combinations thereof.
